(19) United States
(12) Patent Application Publication
(10) Pub. No.: US 2024/0225983 A1
(43) Pub. Date: Jul. 11, 2024

(54) COMPOSITIONS AND METHODS FOR PREVENTING OR REDUCING AUTOXIDATION OF FRAGRANCE AND FOOD OILS

(71) Applicant: Firmenich SA, Satigny (CH)

(72) Inventors: Michael CALANDRA, Plainsboro, NJ (US); Ying WANG, Plainsboro, NJ (US); Dushanee SRIYARATHNE, Plainsboro, NJ (US)

(73) Assignee: Firmenich SA, Satigny (CH)

(21) Appl. No.: 18/557,323

(22) PCT Filed: Apr. 20, 2022
(86) PCT No.: PCT/EP2022/060358

§ 371 Date: Oct. 26, 2023

Related U.S. Application Data
(60) Provisional application No. 63/184,247, filed on May 5, 2021.

(30) Foreign Application Priority Data
May 27, 2021 (EP) 21176136.6

(51) Int. Cl. A61K 8/49; A61K 8/365; A61K 8/37; A61Q 13/00; C11B 5/00

(52) U.S. Cl. CPC A61K 8/498; A61K 8/365; A61K 8/375; A61Q 13/00; C11B 5/0028

(57) ABSTRACT

The present invention relates to 2-oxoacids modified for use as stabilizers in non-polar applications, such as applications that contain food fats or oils, or fragrance oils.

### CROSS REFERENCE TO RELATED APPLICATIONS

This application claims priority to U.S. Provisional Application No. 63/184,247, filed on May 5, 2021, and European Application No. 21176136.6, filed May 27, 2021. The entire contents of these applications are explicitly incorporated herein by this reference.

### FIELD OF THE INVENTION

The present invention provides compositions and methods for controlling autoxidation in fats or oils used in foods, fragrances, and cosmetics (for skin creams, lotions, etc.), and in terpenes and other perfumery raw materials (PRMs).

### BACKGROUND

Autoxidation in fats and oils can result in rancidity, while autoxidation in perfume raw materials (PRMs) can result in skin sensitization issues and/or aroma instability. Many formulated perfumes, body care products, home care products, perfumery raw materials (such as, for example, essential oils, natural extracts, and synthetic ingredients), and food raw materials (such as, for example, fats and oils derived from animal or plant sources, and derivatives thereof, including monoglycerides, diglycerides, lecithins, phosphatidyl ethanolamines, or other phospholipids, and modified triglycerides) undergo oxidation, resulting in the formation of chemical species including peroxides, organic hydroperoxides, peroxyhemiacetals, hemiacetals, acetals, or transesterification products. The chemical species formed as a result of oxidation may alter the organoleptic properties or appearance of perfume ingredients, formulated perfumes, formulated body care products, formulated skin care products, formulated homecare products, essential oils, food raw materials, formulated food products, and natural extracts, or, alternatively, be harmful, irritant, or allergenic. A high peroxide value (POV) of formulated perfumes, body care products, and perfumery raw materials may lead to skin sensitization issues such as, for example, contact dermatitis. An unacceptably high POV can result in a perfumery raw material failing quality control testing and being deemed unusable. An unacceptably high POV can result in a food raw material having an unpleasant rancid taste.

### SUMMARY OF THE INVENTION

The effects of autoxidation in foods, perfumes, and cosmetics are mitigated in the present invention by methods and compositions leading to the chemical consumption of reactive and potentially harmful organic hydroperoxides and/or rancid smelling volatile compounds formed in the autoxidation process. The present invention can not only prevent the formation of rancidity in triglyceride fats/oils, but also reduce the rancidity of fats/oils that have already become rancid; in other words, remediate the rancidity of fats/oils.

The use of 2-oxoacids and related compounds as stabilizers for perfumes, raw materials, and food products is disclosed in US Publication No. 2019/0321274 A1. While 2-oxoacids and/or their salts can be effective in vegetable oils or undiluted fragrance oils, there may be solubility problems in these very hydrophobic matrices due to the ionic nature of a 2-oxoacid salt. The low solubility of 2-oxoacid salts may impose limitations on the applications in which they can be successfully used.

The present invention provides modifications to such compounds, permitting use in non-polar applications, such as applications that contain food fats/oils, and applications in undiluted hydrophobic fragrance oils (fragrance oils that are not dissolved in the typical hydroalcoholic solvent used in eau de toilettes (EDTs)).

The present invention relates to the use of solubilized preparations of α-ketoglutaric acid (AKG) or other 2-oxoacids in solvents that contain the hydroxyl group, especially solvents that have two hydroxyl groups in a 1,2- or a 1,3-orientation on a carbon chain. Non-limiting examples include 1,2-pentylene glycol (1,2-dihydroxypentane); 1,3-butylene glycol (1,3-dihydroxybutane); hexylene glycol (2-Methyl-2,4-pentanediol); diethyl tartrate; a monoglyceride or diglyceride; or mono-/diglyceride mixture derived from any food oil. These solubilized preparations provide alternatives to 2-oxoacid salts in situations where solubility of such salts is problematic. It was surprising that a polar 2-oxoacid, especially the highly polar AKG, was rendered soluble in a hydrophobic media by some unspecified interaction, covalent or otherwise, with a compound containing the polar hydroxyl group. There was an unexpected gain in hydrophobicity from combining the two components.

Methods for controlling autoxidation in fats/oils used in foods, fragrances, and cosmetics, and in terpenes and other perfumery raw materials (PRMs) according to the present invention comprise:
Mixing a 2-oxoacid and hydroxyl group containing compound(s) (SOP) and stirring them, optionally with mild heating, until they form a clear, homogeneous mixture/solution; and
adding the mixed and stirred SOPs to the material to be treated (e.g., fats/oils used in foods and cosmetics, and in terpenes and other perfumery raw materials (PRMs)) and mixing.

### BRIEF DESCRIPTION OF THE FIGURES

FIG. 1 is a graph showing the POV of corn oil treated with ART Compound 1 over the course of days.

FIG. 2 is a graph showing the % Reduction of POV in Corn Oil Treated with ART Compound 1.

FIG. 3 is a graph showing the POV for pure corn oil, DET in corn oil, and AKG/DET in corn oil.

FIG. 4 is a graph showing the % reduction in POV of corn oil treated with 5% 1:10 AKG/DET.

FIG. 5 is a graph showing the POV for undiluted fragrance oil, and fragrance oil with AKG-Aleen5, AKG-BG, AKG-NMDEA, and AKG-DET over days.

FIG. 6 is a graph showing the % reduction in POV for undiluted fragrance oil, and fragrance oil with AKG-Aleen5, AKG-BG, AKG-NMDEA, and AKG-DET over days.

FIG. 7 is a graph showing the POV of untreated EDT and EDT treated with AKG-BG, AKG-diNMDEA, ADG-DET, or AKG-DET at pH 7.

FIG. 8 is a graph showing the POV of is a graph showing the POV of untreated EDT and EDT treated with AKG-BG, AKG-diNMDEA, ADG-DET, or AKG-DET at pH 5.5.

FIG. 9 is graph showing aldehyde concentration (hexanal or 2-nonenal) in a salad dressing treated with phenyl pyruvic acid dimethyldecylammonium salt or untreated.

FIG. 10 is a graph showing the reduction in POV of Treated Corn Oil by ART Compound 2 over time.

### DETAILED DESCRIPTION

The present invention provides methods and compositions for preventing the formation of rancidity in triglyceride fats/oils and/or reducing the rancidity of fats/oils that have already become rancid; in other words, remediate the rancidity of fats/oils. 2-oxoacids (see US Patent Publication No. 2019/0321274) appear to act as aldehyde scavengers, because volatile aldehydes are mostly responsible for rancid aromas in fats/oils.

A “Solubilized 2-Oxoacid Preparation” or “SOP” as used herein refers to 2-oxoacid/hydroxyl containing solvent preparations of the present invention.

SOPs of the present invention include, but are not limited to esters of AKG such as:

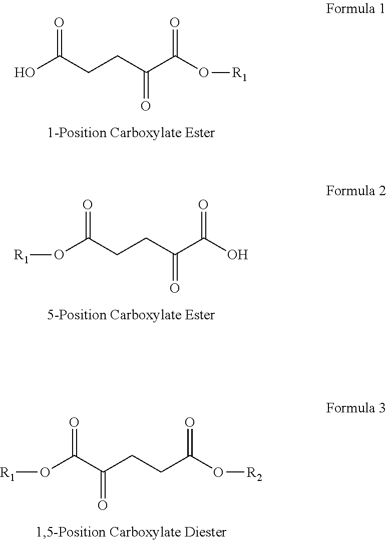

wherein R¹ or R² is the organic moiety (alkyl or aryl) derived from/corresponding to the alcohol R—OH, and R—OH is any alcohol that is sufficiently non-polar to confer enough hydrophobic character to the corresponding ester, relative to the unesterified carboxylate moiety, that the resulting ester would have enhanced solubility in a hydrophobic matrix such as an undiluted fragrance oil, or a food/cooking oil such as sunflower oil. R¹ or R² may be any alkyl or aryl group or combination thereof, which may also contain additional functional groups/substituents including double bonds, hydroxyl groups, ethers, esters, ketones, aldehydes, amides, ketals, or acetals.

Non-limiting examples of alcohols from which the specific R¹ or R² moieties derive include:

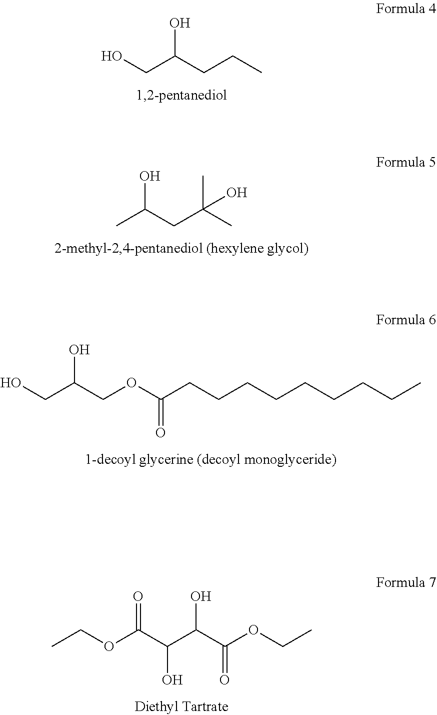

Non-limiting examples of an SOP of the present invention include:

An ester formed from AKG and hexylene glycol:

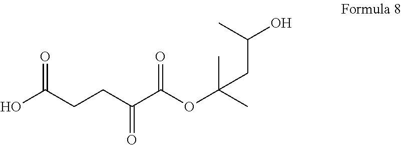 [SMILES: CC(O)CC(C)(C)OC(=O)C(=O)CCC(=O)O]

A diester formed from AKG and hexylene glycol:

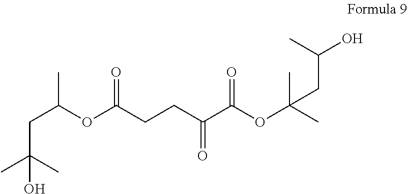 [SMILES: CC(O)CC(C)(C)OC(=O)C(=O)CCC(=O)OC(C)CC(C)(C)O]

A ketal formed from diols that are oriented in a 1,2- or 1,3-positional configuration, such as a ketal formed from AKG and 1,2-pentylene glycol:

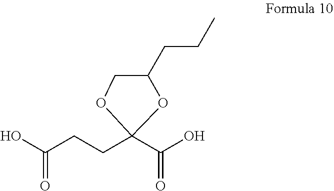 [SMILES: CCCC1COC(CCC(=O)O)(C(=O)O)O1]

One possible isomer of a combined ketal and ester formed from AKG and hexylene glycol:

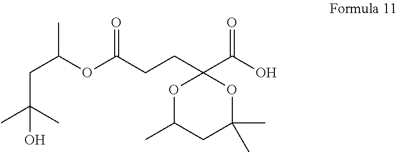 [SMILES: CC(CC(C)(C)O)OC(=O)CCC1(C(=O)O)OC(C)CC(C)(C)O1]

One possible isomer of a combined ketal and diester formed from AKG and hexylene glycol:

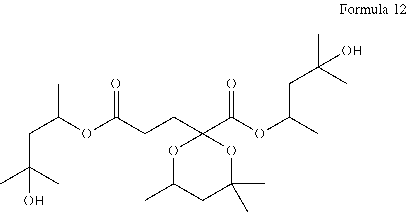 [SMILES: CC(CC(C)(C)O)OC(=O)CCC1(C(=O)OC(C)CC(C)(C)O)OC(C)CC(C)(C)O1]

One possible isomer of an ester formed from AKG and palmitoyl monoglyceride:

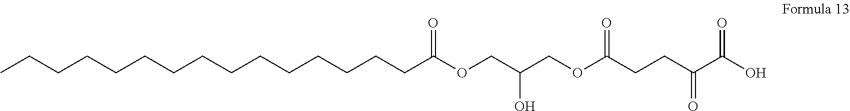 [SMILES: CCCCCCCCCCCCCCCC(=O)OCC(O)COC(=O)CCC(=O)C(=O)O]

A ketal formed from AKG and palmitoyl monoglyceride:

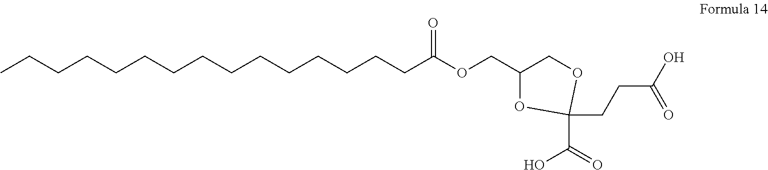 [SMILES: CCCCCCCCCCCCCCCC(=O)OCC1COC(CCC(=O)O)(C(=O)O)O1]

One possible isomer of a combined ketal and ester formed from AKG and palmitoyl monoglyceride:

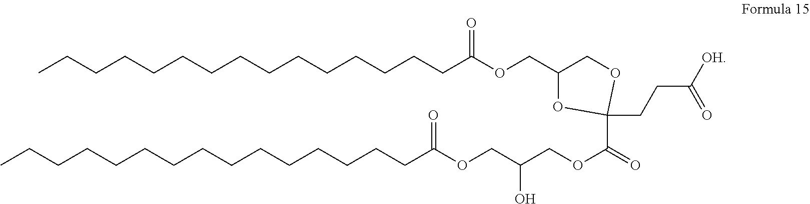 [SMILES: CCCCCCCCCCCCCCCC(=O)OCC(O)COC(=O)C1(CCC(=O)O)OCC(COC(=O)CCCCCCCCCCCCCCC)O1]

As used herein, the term “peroxide value” or “POV” refers to the amount of equivalents of oxidizing potential per 1 kilogram of material. The POV of a material is determined analytically. The term “POV” does not refer to a chemical compound or group of compounds, but may be used interchangeably with the products of autoxidation within a sample that cause a response during a POV test. These autoxidation products differ depending upon the particular material being tested. Many classes of chemical compounds will produce a response during a POV test, including but not limited to organic and inorganic hydroperoxides, organic and inorganic peroxides, peroxyhemiacetals, peroxyhemiketals, and hydrogen peroxide itself.

By way of illustration, one POV test is an iodometric oxidation-reduction titration. All POV-responsive compounds share the property that they are capable of oxidizing the iodide ion to molecular iodine within the time period specified for the test; in fact, the iodide oxidation reaction is the basis for the test. Thus, “POV” is a numerical value that represents the molar sum total of the all the iodide-oxidizing species in a particular sample.

By way of illustration, limonene and linalool are unsaturated terpenes commonly found as major components in many essential oils. Both limonene and linalool are easily oxidized by atmospheric oxygen to form hydroperoxides. The hydroperoxides of limonene and linalool are known to be sensitizers capable of causing contact dermatitis. Consequently, limonene, and natural products containing limonene may only be used as perfumery raw materials when the recommended organic hydroperoxide level is below 20 mmol/L. Similarly, essential oils and isolates derived from the Pinacea family, including Pinus and Abies genera may only be used as perfumery raw materials when the recommended organic hydroperoxide level is below 10 mmol/L.

By way of another illustration, fats and oils, or derivatives thereof, are known to undergo an autoxidation process that leads to unpleasant and unpalatable rancidity. Without intending to be limited to any particular theory, triglyceride hydroperoxides are an intermediate chemical species in the autoxidation process, which further degrade into aldehydes and ketones that produce the rancid aroma.

The POV of a formulated perfume, body care product, cosmetic product, homecare product, perfumery raw material, flavored article, or food raw material may be determined by any method readily selectable by one of ordinary skill in the art. Non limiting examples include, iodometric titration, high-performance liquid chromatography, and the like. An example of an HPLC method for determining the POV of a perfumery raw material is disclosed in Calandra et al., Flavour and Fragr. J. (2015), 30, p 121-130.

Perfumery raw materials include, but are not limited to essential oils, natural extracts, and synthetic ingredients.

The present invention relates to the use of solubilized preparations of α-ketoglutaric acid (AKG) or other 2-oxoacids in solvents that contain a hydroxyl group, especially solvents that have two hydroxyl groups in a 1,2- or a 1,3-orientation on a carbon chain. These solvents must be hydrophobic enough in nature such that they would be easily soluble in the hydrophobic matrices in which one seeks to render treatment. In other words, the solvents would be highly soluble in a food oil or an undiluted fragrance oil. Examples include 1,2 pentylene glycol (1,2 dihydroxypentane), 1,3-butylene glycol (1,3-dihydroxybutane), hexylene glycol (2 Methyl-2,4-pentanediol), diethyl tartrate, or a monoglyceride derived from any food oil. These solubilized preparations provide alternatives to 2-oxoacid salts in situations where solubility of such salts is problematic. According to the present invention, the POV of a formulated perfume, body care product, cosmetic product, homecare product, perfumery raw material, flavored article, or food raw material may be reduced by treating the formulated perfume, body care product, cosmetic product, homecare product, perfumery raw material, flavored article, or food raw material with a SOP.

This invention can provide a benefit over 2-oxoacid salts in very hydrophobic matrices due to the improved solubility of the SOP versus a salt. The exact nature of the interactions between the AKG or other 2-oxoacid and the hydroxyl containing solvent(s) in these SOPs is not currently known. However, the SOPs are presumably not ionic salts, and so can have greater solubility in hydrophobic matrices. Because of the better solubility, SOPs should allow POV remediation treatments to be done in triglyceride oils and their derivatives, and in many PRMs, without having haziness, milky suspensions, or two distinct phases being formed.

SOP compounds according to the present invention react readily with organic hydroperoxides and are compatible organoleptically and toxicologically for use in food, fine fragrance, and body care applications. A hydroperoxide scavenging substance that contains a 2-oxoacid moiety/functional group in the SOP according to the present invention must remain reactive towards hydroperoxides after it is solubilized by the hydrophobic hydroxy-containing solvent.

To form an SOP of the present invention, a solvent must contain a hydroxyl group, or several hydroxyl groups, often in a 1,2- or 1,3-positional configuration, that are able to interact with the 2-oxoacid in such a way that the solvent is capable of solubilizing the 2-oxoacid. The solvent should be readily soluble in hydrophobic media, which will often require an extended carbon chain(s), or ring system(s), or some combination thereof, in the solvent molecule; for example, an alkyl chain of eight to eighteen carbons works.

An SOP of the present invention may be applied as a leave-in ingredient to fragrance raw materials or formulations, or used as an additive in oil containing foods, perfumes, and/or oil-containing skincare products or fragranced skin care products. The SOP may further be used in a manufacturing process to treat raw materials prior to blending them into fully formulated flavor/fragrance oils or consumer products.

An SOP of the present invention may be used in any food preparation that contains fat/oils to prevent the formation of rancidity or remediate existing rancidity, e.g., frying oils. An SOP may further be used in any fragranced skin care product to prevent skin sensitization by terpene hydroperoxides, and to prevent rancidity formation if a triglyceride oil was used (such as in a skin cream). Thus, the present invention may simultaneously serve a dual role in skin care products; to prevent rancidity formation via lipid hydroperoxide scavenging, and to prevent skin sensitization via terpene hydroperoxide scavenging.

In one aspect, an SOP comprises about 0.01% to about 10.0% by weight of the food oil, fragrance oil. In a further aspect the SOP comprises about 0.1% to about 5.0% of the food oil, fragrance oil. In another aspect, the SOP comprises about 0.1% to about 1.0% by weight of the food oil, fragrance oil.

An SOP of the present invention may be used in, for example, in raw materials prior to their being blended into fully formulated flavor or fragrance oils; or in consumer products such as foods and cosmetics (e.g., skin creams, lotions, etc.), oil containing foods (e.g., frying oil, mayonnaise, margarine, and salad dressing), perfumes, oil-containing skincare products or fragranced skin care products.

### EXAMPLES

### Example 1: Corn Oil Treated with a 1:3 Mixture of α-Ketoglutaric Acid and Mono- & Diglycerides Derived from Coconut Oil

Corn Oil Treated with a 1:3 Mixture of α-Ketoglutaric Acid and Mono- & Di-glycerides Derived from Coconut Oil is referred to below as ART Compound 1 (Anti-Rancidity Technology Compound 1).

Preparation of ART Compound 1: 2.0 g of Capmul MCM C8 EP/NF (mono/diglycerides of caprylic acid, ABITEC corporation) was placed into an 10 mL glass vial. This semi solid material was warmed for 20 mins in a 35° C. heat block to melt it. After adding 0.669 g of α-ketoglutaric acid into the vial, the mixture was stirred at room temperature for 2 hrs., producing a white semi-solid. When the vial was again warmed up to 35° C. for 20 mins the mixture melted again. The liquid was stirred at room temperature overnight, then heated to 100° C. for 40 mins. All the AKG finally dissolved to give a light yellow colored liquid, which remained liquid when returned to room temperature.

Treatment of Corn Oil with ART Compound 1 at Various Concentrations:

To 8 mL of corn oil was added the following amounts of ART Compound 1, to produce a series of clear solutions at a range of concentrations, as shown in the table below. The POV of these solutions was measured periodically after standing on the laboratory benchtop at ambient light and temperature conditions.

TABLE 1

| Volume of ART Compound 1 | % v/v in the | Concentration of AKG |
|---|---|---|
| Used (mL) | Corn Oil | in the Corn Oil (ppm) |
| 0.16 | 2 | 6667 |
| 0.08 | 1 | 3333 |
| 0.04 | 0.5 | 1665 |
| 0.016 | 0.2 | 667 |
| 0.008 | 0.1 | 333 |

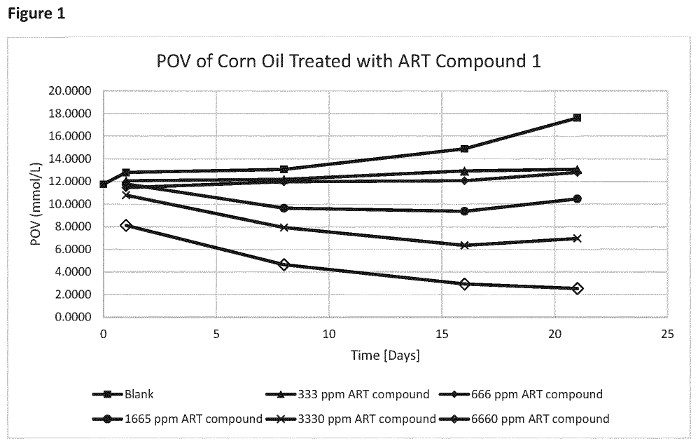

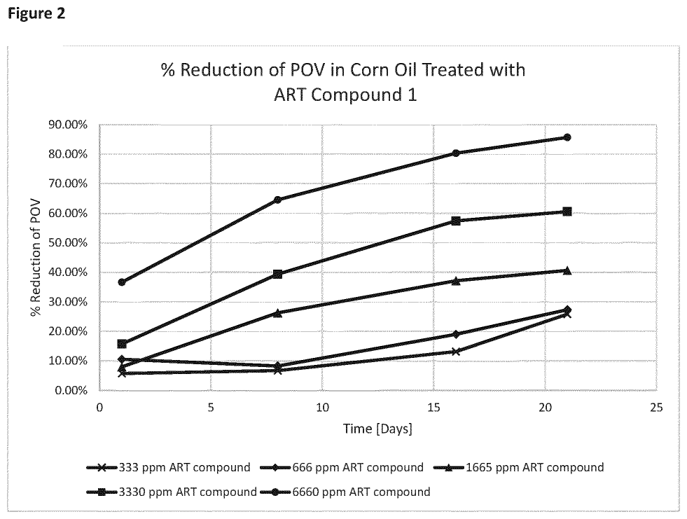

FIG. 1 is a graph showing the POV of corn oil treated with varying amounts of ART Compound 1 over the course of days. FIG. 2 is a graph showing the % Reduction of POV in Corn Oil Treated with varying amounts of ART Compound 1. The graphs show that POV decreased in a dose dependent manner.

### Example 2: Corn Oil Treated with a 1:10 Mixture of α-Ketoglutaric Acid (AKG) and Diethyl Tartrate (DET)

Into a 15 mL glass vial was placed 0.201 gm of AKG and 2.011 gm of DET. The mixture was stirred with a small magnetic stirrer at room temperature over a weekend (approximately 3 days). However, not all of the AKG dissolved, so the vial was placed in a heating block at 90° C. for 15 minutes, and a clear solution was obtained. Upon cooling and standing for several days, no solid precipitated out. This material (0.4 mL) was diluted into 8 mL of corn oil to start the experiment. A sample was also made from just 0.4 mL of DET in 8 mL of corn oil. POV titration measurements were taken periodically of each sample versus untreated corn oil.

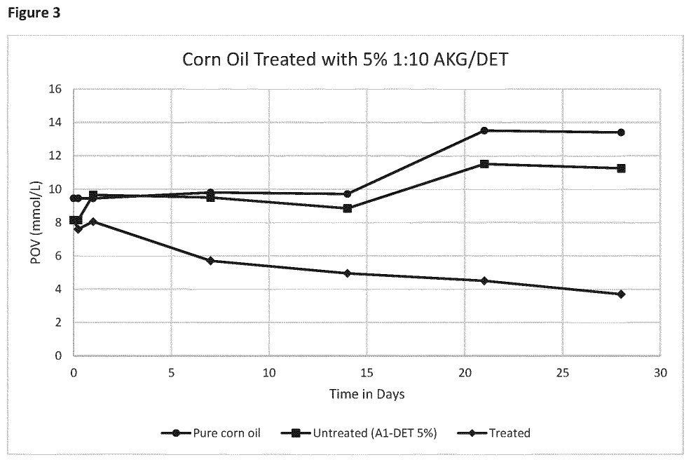

FIG. 3 is a graph showing the POV for pure corn oil, DET in corn oil, and AKG/DET in corn oil.

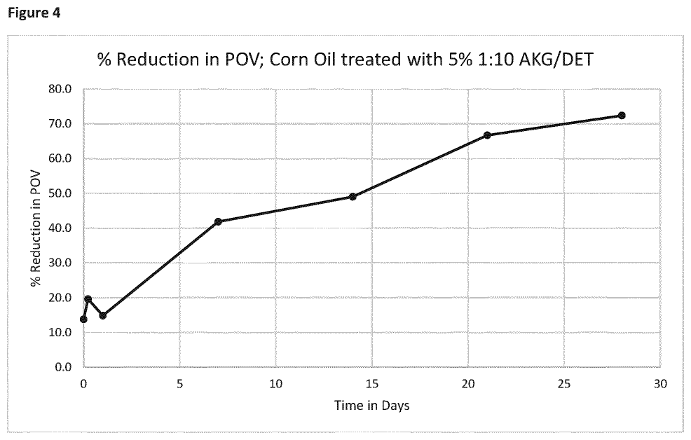

FIG. 4 is a graph showing the % reduction in POV of corn oil treated with 5% 1:10 AKG/DET.

It can be seen that the DET alone, without any AKG solubilized into it, had some measurable activity toward reducing the POV of the corn oil, but it was significantly less than when AKG was also included.

### Example 3: Treatment of a Model Perfume and the Undiluted Perfume Oil

A model perfume oil was made using approximately the following formula:

TABLE 2

| Fragrance oils | Weight (g) | Ratio (w/w) |
|---|---|---|
| Orange oil CP 1122 ARR | 15.0 | 15% |
| Linalool BJ | 10.0 | 10% |
| 2-Buten-1-ol, 2-ethyl-4-(2,2,3- | 10.0 | 10% |
| trimethyl-3-cyclopenten-1-yl)- | | |
| (2-tert-butylcyclohexyl) acetate | 10.0 | 10% |
| 1,3,4,6,7,8-hexahydro-4,6,6,7,8,8,- | 10.0 | 10% |
| hexamethyl-cyclopenta[g]benzopyran | | |
| (IUPAC name: 4,6,6,7,8,8- | | |
| Hexamethyl-1,3,4,6,7,8- | | |
| hexahydrocyclopenta[g]isochromene) | | |
| Tetramethyl | 10.0 | 10% |
| acetyloctahydronaphthalenes (IUPAC | | |
| name: 1-(1,2,3,4,5,6,7,8-octahydro- | | |
| 2,3,8,8,-tetramethyl-2- | | |
| naphthyl)ethan-1-one) | | |
| Methyl dihydrojasmonate (IUPAC | 35.0 | 35% |
| name: Methyl 2-(3-oxo-2- | | |
| pentylcyclopentyl)acetate) | | |

This model perfume oil was diluted and pH adjusted as described below to create two model perfumes, one each at a pH of 7.0 and 5.5.

Prepared model perfume (diluted×5 in 90:10 v/v EtOH/Water):

Two separate bottles (A and B) were prepared (240 mL of each) as described below and the p was measured:
200 mL of 90:10 v/v EtOH/Water and 40 mL of model fragrance oil were mixed and stirred to produce a clear, slightly yellow solution with a pH of 6.16. Fragrance oil concentration: 40 mL/240 mL=16.67% v/v.

The pH of the above prepared model perfume was adjusted to the desired level by using:
Diethylcitrate (DEC), and triethanolamine (TEA, Sigma Aldrich); d=1.124 g/mL, 60 mg TEA=53 uL TEA. A positive displacement pipet was used to transfer the TEA due to its extreme viscosity.
Bottle A—pH 7.0 EDT: To 240 mL of model perfume was added ˜53 μL (60 mg) of TEA, which brought the pH to 7.67. The pH was adjusted to 7.03 by slow addition of DEC under constant stirring while monitoring with a pH meter. The solution remained clear at first, but developed an very slight haze over a few days standing at room temperature.
Bottle B—pH 5.5 EDT: To 240 mL of model perfume was added ˜53 μL (60 mg) of TEA, which brought the pH to 7.77. The pH was adjusted to 5.52 by slow addition of DEC with constant stirring while monitoring with a pH meter. The solution became slightly hazy as a result.

### Example 4: Preparation of SOPs from Various Solvents for Testing in Undiluted Fragrance Oils and Model Perfumes

Preparation of AKG-NMDEA salt (AKG di[N-methyldiethanolammonium] salt); This compound was tested as a known effective treatment, as prepared and described in Flavor & Fragrance Journal, M. Calandra and Y. Wang, 2020, 35:686-694.

Preparation of SOPs from Diethyl Tartrate (DET), 1,3-Butanediol (Butylene Glycol, BG), and 1.2-Pentanediol (A-Leen 5):

AKG-Aleen5 (1:4, mol/mol): To a vial charged with AKG (2.985 g, 0.02 mole) was added 1,2-Pentanediol (Pentylene Glycol, A-Leen5 from Minasolve, 8.426 g, 0.08 mole, 4 equivalents, 26.2% w/w AKG). The mixture was stirred at room temperature overnight to give a clear liquid.

AKG-BG (1:4, mol/mol): To a vial charged with AKG (5.872 g, 0.04 mole) was added 1,3-butylene glycol (Brontide from Genomatica, 14.280 g, 0.16 mole, 4 equivalents, 29.1% w/w AKG). The mixture was stirred at room temperature overnight to give a clear liquid.

AKG-DET (1:5, w/w): To a vial charged with AKG (1.60 g) was added diethyl tartrate (DET, 8.00 g, from Sigma Aldrich, 16.67% w/w AKG). The mixture was stirred at room temperature overnight, but a clear liquid was not obtained. The mixture was then heated to 90° C. for 50 min, and cooled to room temperature to give a clear liquid.

Treatment of Undiluted Fragrance Oil: In 15 ml vials, to separate aliquots of the mixed model fragrance oil (9 mL each aliquot) was added 180 μL (2% v/v) of each SOP, and the samples were vigorously vortexed for ˜1 min, to obtain a homogenous, clear solution (the AKG-NMDEA was an exception in that it was hazy). The resulted samples were then allowed to stand on the laboratory benchtop under ambient light and temperature. POV measurements were taken as a function of time after the addition; the headspace within the vial was refreshed with the ambient atmosphere with each opening of the vial.

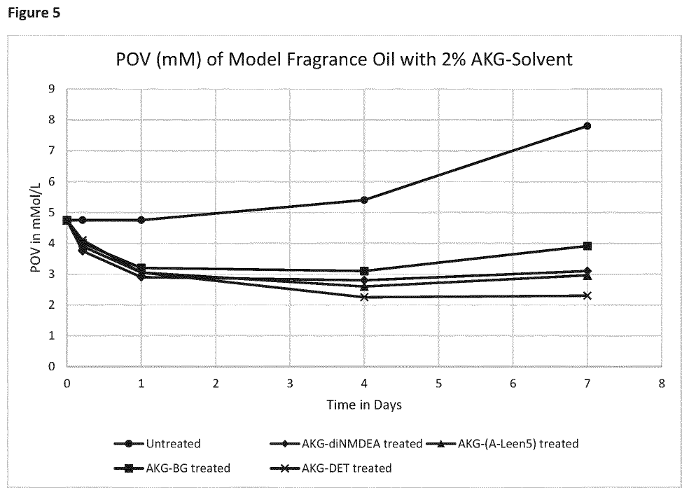

FIG. 5 is a graph showing the POV for undiluted fragrance oil, and fragrance oil with AKG-Aleen5, AKG-BG, AKG-NMDEA, and AKG-DET over days.

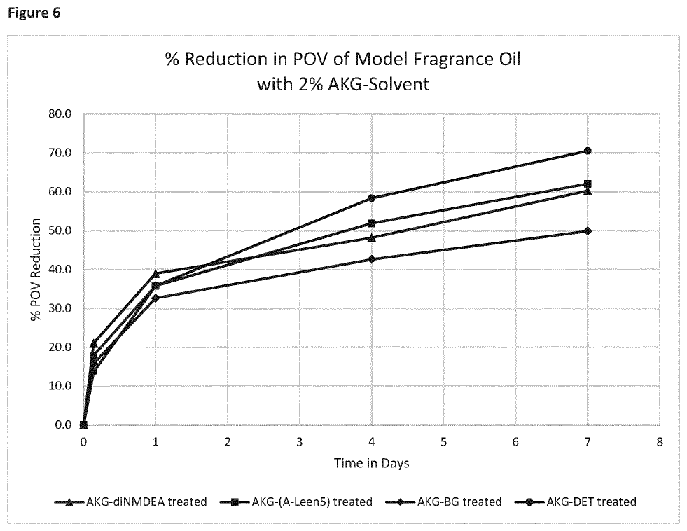

FIG. 6 is a graph showing the % reduction in POV for undiluted fragrance oil, and fragrance oil with AKG-Aleen5, AKG-BG, AKG-NMDEA, and AKG-DET over days.

As shown in FIGS. 5 and 6, POV was reduced in each of the treated fragrance oils relative to the untreated fragrance oil.

### Example 5: Treatment of Model Perfume (Hydroalcoholic EDT Solution)

The SOPs prepared from various solvents, as described above, were dissolved into the model perfume EDT solution according to the chart below. The weight of each SOP used was based on the % (w/w) of AKG in each SOP, so that the final concentration of AKG in the treated model perfume EDT was 0.05% w/v.

TABLE 3

| | | Vol. of | | | |
|---|---|---|---|---|---|
| | % AKG | Treated | Wt. of AKG | Wt. of SOP | |
| AKG-Solvent | (w/w) in | Perfume EDT | (mg) in the | Used | Chemical Name and/ |
| (SOP) | each SOP | (mL) | Added SOP | (mg) | or Brand of Solvent |
| AKG-diNMDEA | 38.00% | 9 mL | 4.5 | 11.8 | N-MethylDiethanolamine |
| AKG-ALeen5 1:4 | 26.2% | 9 mL | 4.5 | 17.2 | 1,2-Pentylene Glycol, |
| (molar equivs.) | | | | | A-Leen5 |
| AKG-BG 1:4 | 29.10% | 9 mL | 4.5 | 15.5 | 1,3-Butylene Glycol, |
| (molar equivs.) | | | | | Brontide (BG) |
| AKG-DET 1:5 | 16.70% | 9 mL | 4.5 | 26.9 | Diethyl Tartrate |
| (w/w) | | | | | (DET) |

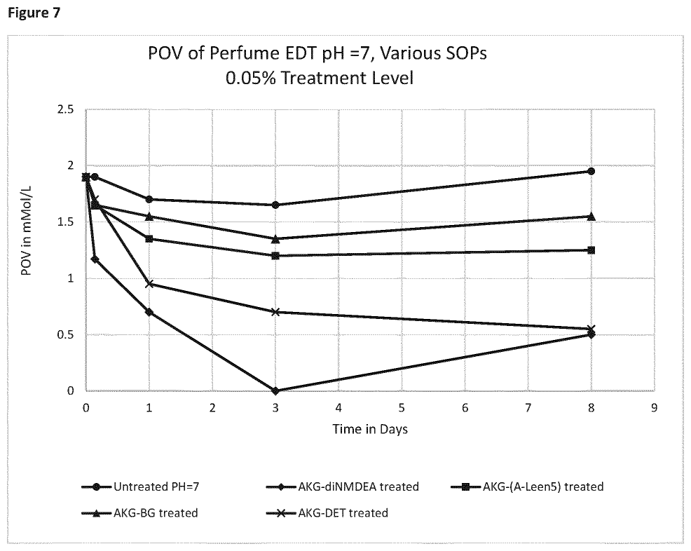

FIG. 7 is a graph showing the POV of untreated EDT and EDT treated with AKG-BG, AKG-diNMDEA, ADG-DET, or AKG-DET at pH 7.

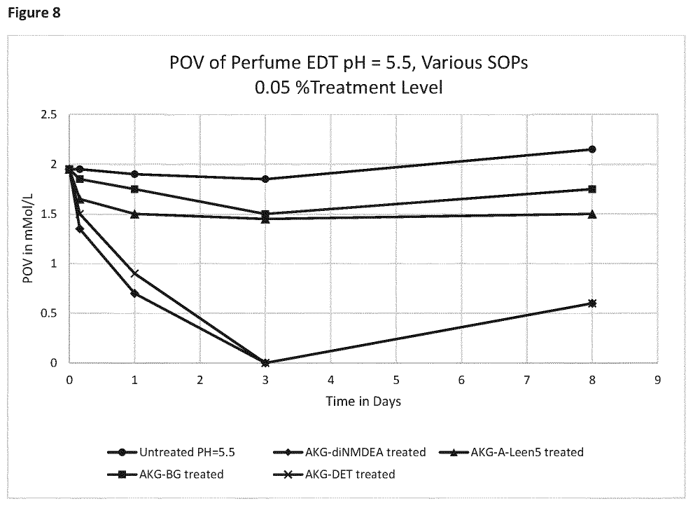

FIG. 8 is a graph showing the POV of is a graph showing the POV of untreated EDT and EDT treated with AKG-BG, AKG-diNMDEA, ADG-DET, or AKG-DET at pH 5.5.

As shown in FIGS. 7 and 8, POV was reduced in each of the treated fragrance oils relative to the untreated EDT.

### Example 6: Aldehyde Scavenging Activity of AKG Salts and AKG-Derived SOPs

Aldehyde levels in corn oil or other foods (such as salad dressing) were measured using the DNPH derivatization followed by HPLC analysis method (J. Ag. Food Chem. Deng, F-Y. et al, (2014) 62, 52, 12545-12552; Food Res. Internat., Cao, J. et al, (2014) 64, 901-907). Alternatively, or in addition to aldehyde measurements by HPLC, organoleptic evaluation to assess the level of rancidity in a sample was done.

Treatment of Salad Dressing with a 2-Oxoacid Salt
Sample: Ranch dressing (chili lime flavor)
2-Oxoacid salt used: Phenyl pyruvic acid dimethyldecylammonium salt
Dosage: ˜2.7% (w/w) (0.4 g of the salt was added to 15 g of salad dressing).
Storage conditions: Room temperature, capped loosely, and mixed periodically.

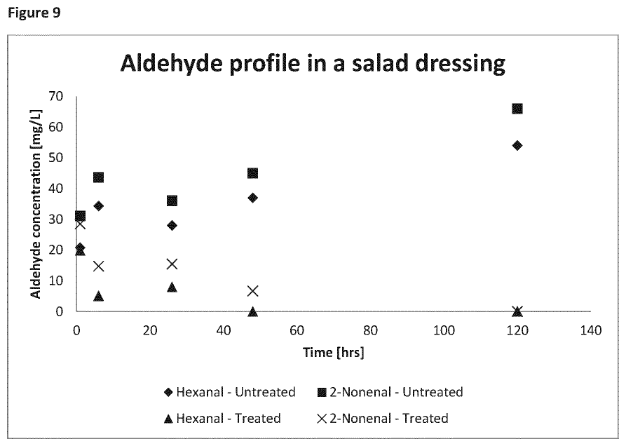

FIG. 9 is graph showing aldehyde concentration (hexanal or 2-nonenal) in a salad dressing treated with phenyl pyruvic acid dimethyldecylammonium salt or untreated. As shown in the graph, aldehyde levels were lower in the treated salad dressing.

For this salad dressing, the dressing had moderately rancid aroma before the treatment, but was significantly lower in rancidity after the treatment.

### Example 7—Performance of the SOPs

The performance of the SOPs were monitored by POV titration as described in Flavor & Fragrance Journal, M. Calandra and Y. Wang, 2020, 35:107-113 or as per the IFRA POV method (see www.ifraorg.org).

SOPs are effective at lowering the POV (removing hydroperoxides) from PRMs, fully formulated fragrance oils, and triglyceride fats/oils. In some cases, rancid smelling volatile compounds are also removed from the triglyceride fats/oils. The SOPs described herein are able to form clear solutions in hydrophobic media such as food oils and a typical model undiluted fragrance oil.

### Example 8—Corn Oil Treated with a 1:5 Mixture of Phenylpyruvic Acid and Mono-& Diglycerides Derived from Coconut Oil

Note: This material is referred to below as ART Compound 2 (Anti-Rancidity Technology Compound 2) for convenience.

Preparation of ART Compound 2: 2.0 g of Capmul MCM C8 EP/NF (mono/diglycerides of caprylic acid, ABITEC corporation) was placed into a 10 mL glass vial. This semi-solid, partially crystalline material was warmed to 35° C. for 20 mins in a heating block until it melted to a clear liquid. After adding 0.404 g of phenylpyruvic acid (PPA), the vial was stirred at room temperature for 10 mins to give a white, hazy mixture. When the vial was warmed to 35° C. for an additional 20 mins, the hazy liquid became transparent. This was further stirred at room temperature for 2 hrs. to give a transparent, yellowish liquid.

### Example 9: Treatment of Corn Oil with ART Compound 2 at Various Concentrations

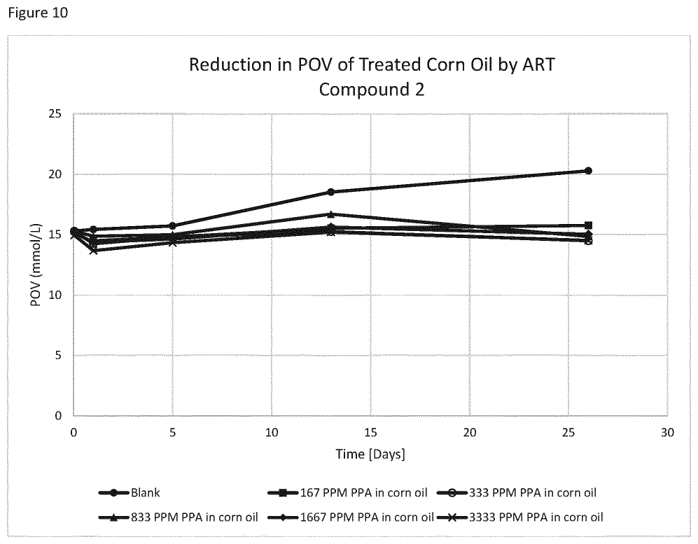

To 16 mL of corn oil was added the following amounts of ART Compound 2, to produce a series of clear solutions as shown in the table below. After adding ART Compound 2 into the oil, the mixture was stirred at RT for 5 mins. All dissolved and no solid precipitation or layer separation was observed for the samples. The POV of these solutions was measured periodically after standing on the laboratory benchtop at ambient light and temperature conditions. See FIG. 10.

TABLE 4

| Volume of ART Compound 2 | % v/v in the | PPM of Phenyl Pyruvic |
|---|---|---|
| Used (mL) | Corn Oil | Acid In the Corn Oil |
| 0.32 | 2 | 3333 |
| 0.16 | 1 | 1667 |
| 0.08 | 0.5 | 833 |
| 0.032 | 0.2 | 333 |
| 0.016 | 0.1 | 167 |

1. A composition comprising a Solubilized 2-oxoacid Preparation (SOP) and a fragrance oil or a food oil.

2. (canceled)

3. The composition of claim 1, wherein the SOP is selected from the group consisting of: 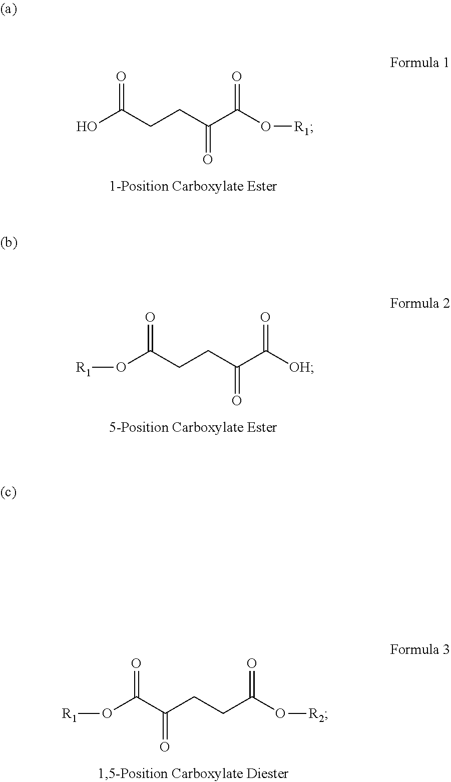
wherein R¹ and R² may be the same or different and are independently selected from the group consisting of: any alkyl or aryl group and combination thereof;
and wherein R¹ and R² may also contain additional functional groups/substituents including double bonds, hydroxyl groups, ethers, esters, ketones, aldehydes, amides, ketals, or acetals.

4. The composition of claim 1, wherein the SOP is selected from the group consisting of: 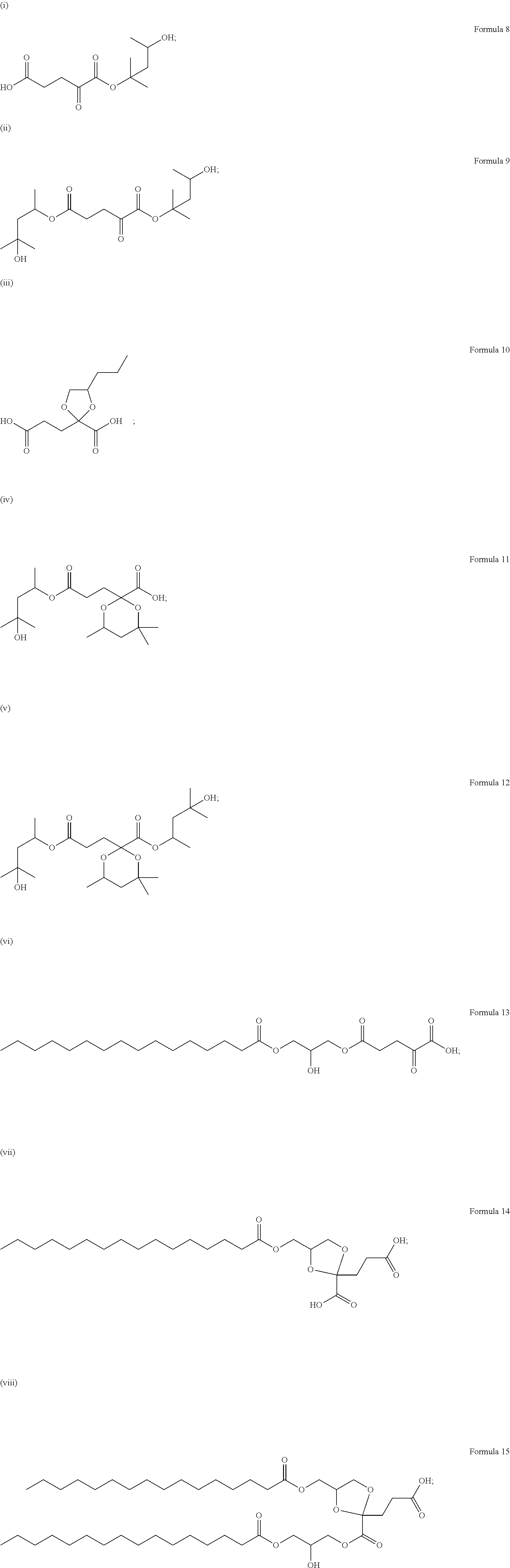
and a combination thereof.

5. A method for preventing the formation of rancidity in triglyceride fats or oils and/or reducing the rancidity of fats or oils that have already become rancid comprising mixing the triglyceride fat or oil with a Solubilized 2-oxoacid Preparation (SOP).

6. A method for reducing the Peroxide Value (POV) of a fragrance oil or a food oil comprising mixing a Solubilized 2-oxoacid Preparation (SOP) and the fragrance oil or the food oil.

7.-10. (canceled)
